(19) United States
(12) Patent Application Publication
(10) Pub. No.: US 2008/0009449 A1
(43) Pub. Date: Jan. 10, 2008

(54) Lignan complex derived from flax seed used for treatment of hypercholesterolemic atherosclerosis

(76) Inventor: Kailash Prasad, Saskatoon (CA)

Correspondence Address: KIRBY EADES GALE BAKER, BOX 3432, STATION D, OTTAWA, ON, K1P 6N9, US

(21) Appl. No.: 11/482,157

(22) Filed: Jul. 7, 2006

(51) Int. Cl. A61K 31/704; A61K 31/717

(52) U.S. Cl. 514/22; 514/25; 514/54

(57) ABSTRACT

A method of treating hypercholesterolemic atherosclerosis in a mammal exhibiting symptoms of the disease. The method comprises administering to the mammal an effective amount of a lignan complex derived from flax and containing about 34 to 38% by weight of secoisolariciresinol diglucoside. The invention also relates to compositions and uses of the complex suitable for the treatment of the disease and related conditions.

### BACKGROUND OF THE INVENTION

I. FIELD OF THE INVENTION

This invention relates to the use of lignan complex isolated from flax for the treatment of hypercholesterolemic atherosclerosis in humans and animals.

II. BACKGROUND ART

Hypercholesterolemia is a major risk factor for atherosclerosis (narrowing of the artery due to deposition of fat in the arterial wall) and related occlusive vascular diseases such as heart attack, stroke and other peripheral vascular diseases. Heart disease is the number one killer. Hypercholesterolemic atherosclerosis has been reported to be associated with oxidative stress (increase in levels of reactive oxygen species (ROS), production of ROS by polymorphonuclear leukocytes as assessed by chemiluminescence (PMNL-CL), and a decrease in the antioxidant reserve) (References 1-4, see list of References at the end of this description). Pretreatment with antioxidants (vitamin E, probucol, garlic, purpurogallin, secoisolariciresinol diglucoside) prevents the effects of hypercholesterolemia (References 1, 2, 4-6). Flax (especially flaxseed) is a rich source of α-linolenic acid and the richest source of plant lignans. Flaxseed has been shown to be effective in preventing the development of hypercholesterolemic atherosclerosis without lowering serum levels of cholesterol (Reference 7). Using flaxseed which has very low α-linolenic acid has shown that the antiatherogenic activity of flaxseed is not due to α-linolenic acid but may be due to the lignan component of flaxmeal (Reference 8).

Presently, the treatment of hypercholesterolemia and hypercholesterolemic atherosclerosis is to reduce hypercholesterolemia by using various lipid lowering agents such as bile acid sequestrant (cholestyramine, colestipol), nicotinic acid, HMG-CoA reductase inhibitor (lovastatin, pravastatin, simvastatin, fluvastatin and atorvastatin) and gemfibrozil. Recently probucol which has both antioxidant and lipid lowering activity and vitamin E which has antioxidant activity have been used to prevent atherosclerosis and restenosis.

Drugs used for lowering serum lipids and for treatment of atherosclerosis (heart attack and stroke) have many side effects and are expensive. Fibric acid derivatives (gemfibrozil) produces gall stones, myopathy and hepatomegaly. Nicotinic acid produces gastrointestinal symptoms, flushing, hyperglycemia, hepatic dysfunction, elevated uric acid, abnormal glucose tolerance, and skin rash. Bile acid sequestrant (cholestyramine, colestipol) produces gastrointestinal symptoms, and high serum levels of very low density-lipoprotein (VLDL). HMG-CoA reductase inhibitors (statin) produce gastrointestinal symptoms, myopathy and hepatotoxicity. Probucol produces diarrhea and decreases the serum levels of HDL (good cholesterol).

Prasad, U.S. Pat. No. 5,846,944, describes the use of secoisolariciresinol diglucoside (SDG), isolated from flaxseed, for reducing hypercholesterolemic atheroscleorsis and reducing serum cholesterol. However, isolating SDG from flaxseed is a relatively expensive procedure.

In Westcott et al., U.S. Pat. No. 6,264,853, a new lignan complex is described which has been isolated from flaxseed. This lignan complex typically contains SDG (35%), cinnamic acid glycosides and hydroxymethyl glutaric acid. Only a simple procedure is required to isolate this lignan complex from flaxseed.

In Prasad, U.S. Pat. No. 6,673,773, a method for preventing hypercholesterolemic atheroschlerosis is disclosed.

One purpose of the present invention, at least in preferred forms, is to provide a method of treating hypercholesterolemic atheroschlerosis in subjects that already have symptoms of the disease.

### SUMMARY OF THE INVENTION

One exemplary aspect of the invention provides a method of treating hypercholesterolemic atherosclerosis in a mammal (human or animal) exhibiting symptoms thereof. The method comprises administering to the subject an effective amount of a lignan complex derived from flax and containing about 34 to 38% by weight of secoisolariciresinol diglucoside (SDG), and preferably about 15 to 21% by weight cinnamic acid glucosides and about 9.6 to 11% by weight hydroxymethylglutaric acid. The cinnamic acid glucosides preferably include coumaric acid glucoside and ferulic acid glucoside.

According to another exemplary aspect of the invention, there is provided a composition comprising a lignan complex derived from flax capable of reducing serum cholesterol, raising high-density lipoprotein cholesterol, reducing hypercholesterolemic artherosclerosis, or any combination thereof, when orally admisistered to a mammal at a daily does of 20-60 mg per kg body weight of the mammal.

Yet another exemplary embodiment of the invention provides a process comprising obtaining a lignan complex comprising secoisolariciresinol diglucloside, cinnamic acid glycosides and hyroxymethyl glutaric acid from a plant; wherein a component of the lignan complex has a molecular weight of at least 30,000; and placing the lignan complex in a composition suitable for oral administration to a mammal.

Yet another exemplary embodiment of the invention provides a composition comprising: a lignan complex obtained from flax and comprising secoisolariciresinol diglucoside (SDG), cinnamic acid glucosides, and hydroxymethylglutaric acid; wherein a component of the lignan complex has a molecular weight of at least 30,000; wherein the lignan complex is effective in at least one of reducing serum cholesterol, raising high-density lipoprotein cholesterol, and reducing hypercholesterolemic artherosclerosis when orally administered to a rabbit at a daily dose of 40 mg/kg of body weight of the rabbit for a period of two months.

### BRIEF DESCRIPTION OF THE DRAWINGS

FIG. 1 is a representative photograph of intimal surface of aorta from rabbits of four experimental groups of slowing of progression study showing Sudan IV—stainable lipid deposit. Note marked brick red lipid deposits in groups II, III and IV.

FIG. 2 is a graph showing the extent of atherosclerosis in aortas of the four experimental groups referred to above.

FIG. 3 shows photographs of the intimal surfaces of aortas of rabbits from 4 experimental groups of a regression study showing Sudan IV—stainable lipid deposits.

FIG. 4 is a graph showing the extent of atherosclerosis in the aortas of the four experimental groups referred to above.

### DETAILED DESCRIPTION OF THE PREFERRED EMBODIMENTS

The effectiveness of lignan complex from flax for the prevention of atherosclerosis in subjects that show no symptoms of the disease has previously been demonstrated by the inventor of the present invention in U.S. Pat. No. 6,673,773, the disclosure of which is incorporated herein by reference.

The inventor has now established that lignan complex is not only effective in preventing the development of hypercholesterolemic atheroschlerosis in healthy mammals, but can actually slow the progression of the disease once it has developed in a subject

Lignan complex from flax, and a method for its extraction, is described in U.S. Pat. No. 6,264,853, issued Jul. 24, 2001, the disclosure of which is incorporated herein by reference. The process may comprise obtaining an aqueous aliphatic alcohol extract of commercial flax, e.g. flaxseed or flax meal. The extract is then subjected to ultrafiltration, whereby low molecular weight species remain with the filtrate and higher molecular weight species are retained. By proper selection of the filtration medium, it is possible to retain a lignan complex in substantially pure form comprising secoisolariciresinol diglucoside (SDG), cinnamic acid glucosides (CAG), and hydroxymethylglutaric acid (HMGA). The ultrafiltration is preferably carried out for a size exclusion of 30,000 Daltons or greater, generally in the range of 30,000 to 100,000 Daltons, and more preferably 30,000 to 50,000 Daltons.

The complex used according to the present invention typically contains 34 to 38% by weight of secoisolariciresinol diglucoside (SDG), and optionally 15 to 21% by weight cinnamic acid glucosides (CAG), and 9.6 to 11% by weight hydroxymethylglutaric acid (HMGA). The CAG component typically includes coumaric acid glucoside and/or ferulic acid glucoside, normally present in the complex in amounts of about 9.5 to 16.0% by weight coumaric acid glucoside and 4.5 to 5.0% by weight ferulic acid glucoside.

The complex typically contains about 59 to 70% by weight of the three stated ingredients, the balance comprising protein, ash and water of crystallization.

In the present invention, the complex is preferably employed at a purity of 95% by weight or more.

As noted above, the lignan complex has been found effective for slowing the progression of atherosclerosis and preventing the progression of atherosclerosis that occurs after removal of high cholesterol diet (normalizing the serum cholesterol), and hence may be useful for the treatment of related diseases, such as coronary artery disease, stroke, intermittent claudication, restenosis following coronary artery percutaneous intervention (PCI), coronary bypass grafts closure, and other peripheral vascular disease, as well as reducing complications associated with such diseases.

The lignan complex may be administered orally in any suitable form, e.g. as a medicament for suitable for treating atherosclerosis in a mammal, preferably mixed with a pharmaceutically-acceptable diluent or carrier, optionally prepared, for example, as a tablet, capsule or food. Suitable dosages may be determined by trail and experimentation, but dosages of 20-60 mg per kg body weight of the mammal are normally most effective, particularly when the mammal is a rabbit.

The invention is further illustrated by the following non-limiting Examples.

### EXAMPLES

### Methods:

The studies below were conducted in New Zealand white female rabbits weighing between 1.2 and 1.5 g. (6 to 8 wks old) after a one-week period of acclimatization. The rabbits were housed individually under constant climate conditions (temperature 20 to 22° C., relative air humidity 50±5%). The rabbits were housed under a 12-hour light and 12-hour dark cycle. The studies were approved by the Ethics Committee of the University of Saskatchewan, Saskatchewan, Canada, and the animal care was according to approved standard for Laboratory Animal Care. Food and water were provided ad libitum.

### Experimental Protocol

(i) For the study of the slowing of progression of atherosclerosis:
Rabbits in this study were divided into 4 groups.
   Group I (n=6): control (regular diet).
   Group II (n=5): 0.25% cholesterol diet for 2 months.
   Group III (n=6): 0.25% cholesterol diet for 4 months.
   Group IV (n=6): 0.25% cholesterol diet for 2 months, followed by 0.25% cholesterol diet and lignan complex (40 mg/kg body weight per day, orally) for an additional two months.

(ii) For the study of the regression of atherosclerosis:
Rabbits in this study were assigned to 4 groups.
   Group I (n=6): control (regular diet)
   Group II (n=5): 0.25% cholesterol diet for 2 months.
   Group III (n=11): 0.25% cholesterol diet for 2 months followed by regular diet for 4 months.
   Group IV (n=12): 0.25% cholesterol diet for 2 months followed by regular diet and lignan complex (40 mg/kg body weight per day, orally) for additional 4 months.

At the end of the protocol, rabbits were anesthetized with pentobarbital sodium (40 mg/kg, intravenously) and the aortas were removed for the assessment of atherosclerotic changes. The assessment of atherosclerotic changes in the aorta was made by using Herxheimer's solution containing Sudan® IV for lipid staining (1). Photographs of the stained intimal surface of the aorta were taken and color slides were prepared. The slides were projected on a Caramate® Projector screen with a grid in mm². The extent of atherosclerosis was expressed as a percentage of total intimal surface area.

### Statistical Analysis:

Results are expressed as mean±SEM. The Kruskal-Wallis test was used to determine the differences in the atherosclerotic changes in the four groups. The Mann-Whitney U test was used to determine the significance of differences between any two groups. Type-1 error for multiple comparison was controlled by the Bonferroni correction.

### Results:

### Slowing of Progression of Atherosclerosis

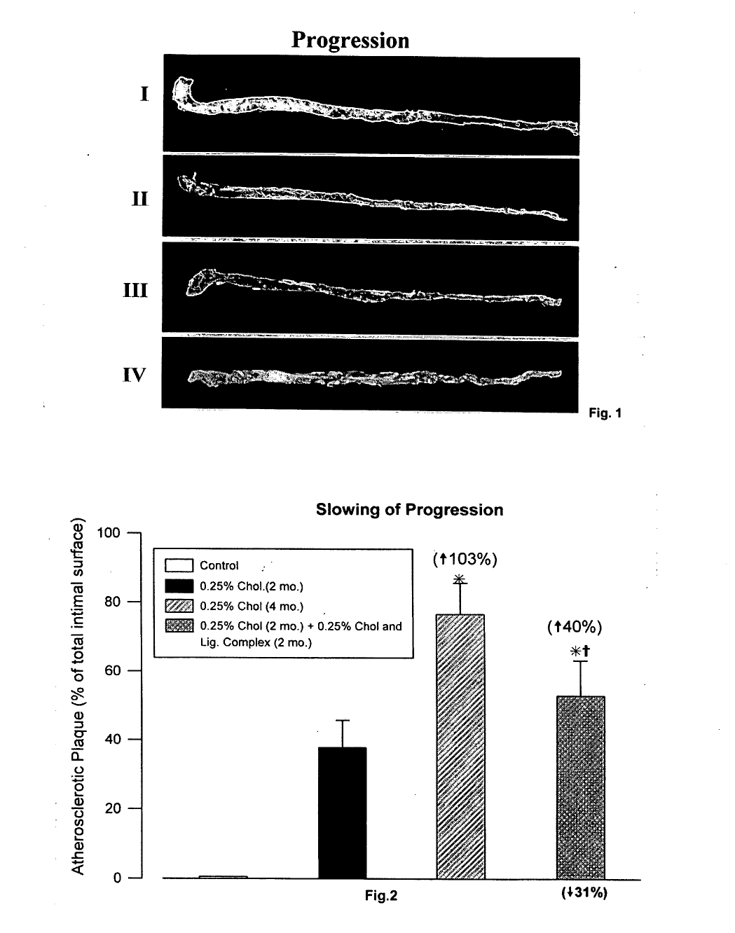

This study was conducted to see if lignan complex would slow the progression of atherosclerosis. Representative photographs of the atherosclerotic changes in the intimal surface of the aortas of the four groups are shown in FIG. 1 and the results are summarized in FIG. 2 (the results are expressed as mean±SEM).

The original photographs for FIG. 1 included brick red areas showing lipid deposits for Groups II, III and IV. In the black and white photographs of the accompanying drawings, the lipid deposits appear as grey areas.

FIG. 2 shows the extent of atherosclerosis in the aortas of the four experimental groups. The results are expressed as mean±SEM. There was no atherosclerosis in the control group (the small value shown in the figure is just to locate the control group). The abbreviations shown in FIG. 2 represent the following:
Chol.=cholesterol
mo.=months
Lig.=lignan.

The numerical values in brackes above the bars in FIG. 2 shows the percentage increase in the respective groups compared to the group fed 0.25% cholesterol for 2 months. The numerical values in brackets below the bar shows the percentage decrease in the group compared to the group fed 0.25% cholesterol for four months. In FIG. 2:

* p<0.05, 0.25% cholesterol (2 months) vs. 0.25% cholesterol (4 months), or 0.25% cholesterol (2 months)+0.25% cholesterol and lignan complex (2 months).

† p<0.25, 0.25% cholesterol (4 months) vs. 0.25% cholesterol (2 months)+0.25% cholesterol and lignan complex (2 months).

The intimal surface of the aorta of Groups II, III and IV were covered with atherosclerotic plaques to the extent of 37.76±7.96% and 76.6±9.04% and 52.95±10.29%, respectively. As expected 0.25% cholesterol diet for four months produced a 103% greater extent of atherosclerosis than a similar diet for two months. The atherosclerotic process progressed with hypercholesterolemic diet. The rabbits in group IV, which received 0.25% cholesterol and lignan complex for two months following a 0.25% cholesterol diet for two months, had a lesser extent of atherosclerotic plaques compared to Group III that received 0.25% cholesterol for 4 months (52.95±10.3% vs. 76.59±9.04%). Lignan complex group (Group IV) had 40% greater atherosclerosis as compared to Group II, but 31% less than that of Group III. Lignan slowed the progression of atherosclerosis by 31%. These results suggest that lignan complex slows the progression of atherosclerosis.

### Regresssion of Atherosclerosis

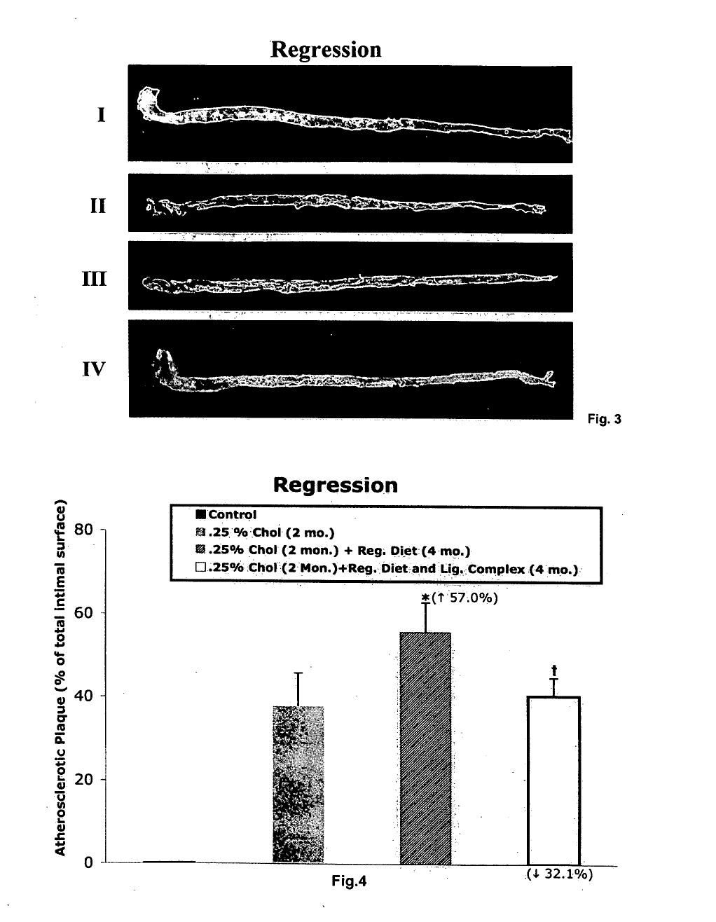

This study was conducted to determine if lignan complex can produce regression of already-developed atherosclerosis. The representative photographs of the atherosclerotic changes in the intimal surface of the aortas from the four groups are shown in FIG. 3 and the results are summarized in FIG. 4 (the results are expressed as mean±SEM).

The original photographs for FIG. 3 included brick red areas showing lipid deposits for Groups II, III and IV. In the black and white photographs of the accompanying drawings, the lipid deposits appear as grey areas.

The results shown in FIG. 4 are expressed as mean±SEM. The abbreviations used in FIG. 4 represent the following:
Chol.=cholesterol
mo.=months
Lig.=lignan
Reg.=regular
↑=increase
↓=decrease.

The numerical values in brackets above the bars in FIG. 4 show the percentage changes in the respective groups compared to the group fed 0.25% cholesterol for 2 months. The numerical values in brackets below the bar shows the percentage change in the group compared to the group fed 0.25% cholesterol for two months and a regular diet for four months.

In FIG. 4:

* p<0.05, 0.25% cholesterol (2 months) vs. 0.25% cholesterol (2 months)+regular diet (4 months), or 0.25% cholesterol (2 months)+regular diet and Lignan complex (4 months)

† p<0.05, 0.25% cholesterol (2 months)+regular diet (4 months) vs. 0.25% cholesterol (2 months)+regular diet and lignan complex (4 months).

The atherosclerotic plaques were absent in the group of rabbits (Group I) on a regular diet. It was found that 37.75±7.96% of the intimal surface of the aortas of rabbits fed on 0.25% cholesterol for two months was covered with atherosclerotic plaques. The extent of atherosclerosis in group of rabbits on a regular diet for 4 months following 0.25% cholesterol diet was 57.0% greater than those on 0.25% cholesterol diet for 2 months (atherosclerosis 59.29±6.71 vs. 37.76±7.96%). This shows that the atherosclerotic process continued even on regular diet for 4 months following a 0.25% cholesterol diet. The extent of atherosclerosis in the group of rabbits on lignan complex and a regular diet (Group IV) was not significantly different from those of group II (40.26±4.42 vs. 37.76±7.96), but was 32.1% lower compared to Group III. These results suggest that the lignan complex did not produce regression of already present atherosclerotic lesions, however it completely prevented the progression of atherosclerosis that developed after removal of high cholesterol diet (normalization of serum lipids).

### CONCLUSION

The results suggest that lignan complex is very effective in slowing the progression (28-31%) of atherosclerosis. It did not produce regression of atherosclerosis. However, it completely prevented the further development of atherosclerosis that occurs after removal of a high cholesterol diet.

### REFERENCES

1. Prasad K, Kalra J. Oxygen free radicals and hypercholesterolemic atherosclerosis: effect of vitamin E. Am Heat J 1993;125:958-73.
2. Prasad K, Kalra J, Lee P.; Oxygen free radicals as a mechanisms of hypercholesterolemic atherosclerosis: effects of probucol. Int J Angiol 1994;3 :10-12.
3. Steinberg D. Antioxidants in the prevention of human atherosclerosis. Circulation 1992;85:2338-45.
4. Prasad K. Reduction of serum cholesterol and hypercholesterolemic atherosclerosis in rabbits by secoisolariciresinol diglucoside isolated from flaxseed. Circulation 1999;99:1355-62.
5. Prasad K, Mantha S V, Kalra J, Kapoor R, Kamalarajan B R C. Purpurogallin in the prevention of hypercholesterolemic atherosclerosis. Int J Angiol 1997:6:157-66.
6. Prasad K, Mantha S V, Kalra J, Lee P. Prevention of hypercholesterolemic atherosclerosis by garlic, an antioxidant. J Cardiovasc Pharmacol Ther 1997;2:309-20.
7. Prasad K. Dietary flaxseed in the prevention of hypercholesterolemic atherosclerosis. Atherosclerosis 1997; 132:69-75.
8. Prasad K, Mantha S V, Muir A D, Westcott N D. Reduction of hypercholesterolemic atherosclerosis by CDC-flaxseed with very low alpha-linolenic acid. Atherosclerosis 1998;136:367-375.

1. A method of treating hypercholesterolemic atherosclerosis in a mammal exhibiting symptoms thereof, which method comprises administering to said mammal an effective amount of a lignan complex derived from flax and containing about 34 to 38% by weight of secoisolariciresinol diglucoside.

2. The method of claim 1, wherein said complex further comprises about 15 to 21% by weight cinnamic acid glucosides and about 9.6 to 11% by weight hydroxymethylglutaric acid.

3. The method of claim 2, wherein the cinnamic acid glucosides include coumaric acid glucoside and ferulic acid glucoside.

4. A composition comprising a lignan complex derived from flax capable of reducing serum cholesterol, raising high-density lipoprotein cholesterol, reducing hypercholesterolemic artherosclerosis, or any combination thereof when orally admisistered to a mammal at a daily does of 20-60 mg per kg body weight of the mammal.

5. The composition of claim 4, wherein the lignan complex comprises a compound selected from the group consisting of secoisolariciresinol diglucoside, cinnamic acid glycosides, hydroxymethyl glutaric acid, and any combinations thereof.

6. The composition of claim 4, wherein a portion of the lignan complex has a molecular weight of at least 30,000.

7. The composition of claim 4, wherein the composition is selected from the group consisting of a tablet, a capsule, and a food.

8. The composition of claim 4, further comprising a compound selected from the group consisting of protein, ash and water of crystallization.

9. The composition of claim 4, wherein the lignan complex is of a flax origin.

10. The composition of claim 4, wherein the mammal is a rabbit.

11. The composition of claim 4, further comprising a diluent or carrier.

12. The composition of claim 4, wherein the lignan complex comprises 34 to 38% by weight secoisolariciresinol diglucoside.

13. A process, comprising:
obtaining a lignan complex comprising secoisolariciresinol diglucloside, cinnamic acid glycosides and hyroxymethyl glutaric acid from a plant;
wherein a component of the lignan complex has a molecular weight of at least 30,000; and
placing the lignan complex in a composition suitable for oral administration to a mammal.

14. The process of claim 13, further comprising orally administering the composition to a mammal.

15. The process of claim 13, wherein the composition further comprises a compound selected from the group consisting of protein, ash and water of crystallization.

16. The process of claim 13, wherein the lignan complex is effective in at least one of reducing serum cholesterol, raising high-density lipoprotein cholesterol, and reducing hypercholesterolemic artherosclerosis when orally administered to the mammal at a does of 20-60 mg per kg body weight of the mammal.

17. The process of claim 13, wherein the plant is flax.

18. The process of claim 13, wherein the composition is selected from the group consisting of a tablet, a capsule, and a food.

19. The process of claim 16, wherein the mammal is a rabbit.

20. The process of claim 13, wherein the lignan complex is of a flaxseed origin and contains about 34 to 38% by weight of secoisolariciresinol diglucoside (SDG), about 15 to 21 % by weight cinnamic acid glucosides, and about 9.6 to 11% by weight hydroxymethylglutaric acid.

21. A composition comprising:
a lignan complex obtained from flax and comprising secoisolariciresinol diglucoside (SDG), cinnamic acid glucosides, and hydroxymethylglutaric acid;
wherein a component of the lignan complex has a molecular weight of at least 30,000;
wherein the lignan complex is effective in at least one of reducing serum cholesterol, raising high-density lipoprotein cholesterol, and reducing hypercholesterolemic artherosclerosis when orally administered to a rabbit at a daily dose of 40 mg/kg of body weight of the rabbit for a period of two months.

22. The composition of claim 21, further comprising a diluent or a carrier.

23. The composition of claim 21, wherein the composition is selected from the group consisting of a tablet, a capsule, and a food.

24. Use of a lignan complex derived from flax for the treatment of hypercholesterolemic atherosclerosis in a mammal.

25. Use of claim 24, wherein the mammal is a rabbit.

26. Use of claim 24, wherein the complex comprises about 34 to 38% by weight of hypercholesterolemic atherosclerosis.

27. Use of a lignan complex derived from flax for the preparation of a medicament suitable to the treatment of hypercholesterolemic atherosclerosis in a mammal.
